(19) United States
(12) Patent Application Publication
(10) Pub. No.: US 2009/0305272 A1
(43) Pub. Date: Dec. 10, 2009

(54) METHOD OF CHARACTERIZING ENDOGENOUS POLYNUCLEOTIDE-POLYPEPTIDE INTERACTIONS

(76) Inventors: Zhengne Wang, Beachwood, OH (US); Xiaodong Zhang, Hubei (CN)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/348,630

(22) Filed: Jan. 5, 2009

Related U.S. Application Data
(60) Provisional application No. 61/019,017, filed on Jan. 4, 2008.

(51) Int. Cl. C12Q 1/68; G01N 33/53; C12N 15/63

(52) U.S. Cl. 435/6; 435/7.1; 435/320.1

(57) ABSTRACT

A method for characterizing an endogenous polypeptide includes introducing epitope tag-encoding polynucleotide into an endogenous locus of a somatic cell by homogenous recombination mediated knock-in so that an epitope tagged endogenous polypeptide is expressed by the cell, and characterizing the epitope tagged endogenous polypeptide using an immunoassay.

### RELATED APPLICATION

This application claims priority from U.S. Provisional Application No. 61/019,017, filed Jan. 4, 2008, the subject matter, which is incorporated herein by reference.

### GOVERNMENT FUNDING

This invention was made with government support under Grant No. 1R01HG004722-01 awarded by The National Institute of Health. The United States government has certain rights in the invention.

### TECHNICAL FIELD

The present invention relates generally to a targeting vector for genetically modifying somatic cells, and more particularly to a method of expressing an endogenous gene in a chromosomal locus to characterize endogenous polypeptide and endogenous polynucleotide interactions.

### BACKGROUND OF THE INVENTION

The polynucleotide sequence of the human genome encodes approximately 25,000 proteins. Characterizing all 25,000 depends on the availability of high quality antibodies that can be used for multiple applications, such as Western blot, immunofluorescence and immunoprecipitation. For analysis of transcription factors and other DNA-binding proteins, chromatin immunoprecipitation-grade (ChIP-grade) antibodies capable of immunoprecipitating a protein of interest within the context of chromatin are most often desired. Notwithstanding, ChIP-grade antibodies exist for a small fraction of chromatin-associated proteins. This is particularly problematic for ChIP-chip or ChIP-sequencing studies, where the use of more than one antibody is highly recommended.

The antibody problem can be circumvented by generating cell lines that stably express epitope-tagged proteins recognizable by available antibodies. This approach, however, is far from ideal given that expression is no longer endogenous, which may complicate interpretation of results. Additionally, the construction of recombinant plasmids containing both full-length cDNA and epitope sequences can be cumbersome, particularly for proteins encoded by large transcripts. In principle, this problem can be circumvented by utilizing ectopically-expressed, epitope-tagged proteins recognizable by well characterized antibodies. The problem remains, however, because such expression is no longer endogenous.

### SUMMARY OF THE INVENTION

The present invention relates to a method of determining polynucleotide binding sites of an endogenous polypeptide of a somatic cell. The method includes knocking-in an epitope tag-encoding polynucleotide into an endogenous locus of the somatic cell so that an epitope tagged endogenous polypeptide is expressed by the cell and binds to the endogenous polynucleotide. The tagged polypeptide and the endogenous polynucleotide are then immunoprecipitated with an antibody that is specific to the tag. The identity of the immunoprecipitated polynucleotide is then determined.

In an aspect of the invention, the polynucleotide can include DNA of a genome of the somatic cell and the polypeptide can include a transcription factor. The identity of the immunoprecipitated polynucleotide can be determined using at least one of a polynucleotide microarray or PCR.

In another aspect, the epitope tagged endogenous polynucleotide can be knocked-in by homologous recombination mediated knock-in. The homologous recombination mediated knock-in can be performed by transfecting the somatic cell with a targeting vector. The targeting vector can include a delivery vehicle linked to a modification cassette. The modification cassette can include the epitope tag-encoding polynucleotide.

In a further aspect, the targeting vector can be constructed to genetically modify an endogenous target gene locus in the somatic cell. The modification cassette can further include first and second multiple cloning sites (MCSs) and a polynucleotide sequence encoding a selectable marker conferring drug resistance. The targeting vector can also be packaged for delivery to the somatic cell.

In another aspect, the targeting vector can be constructed by ligating the delivery vehicle with the modification cassette and inserting the polynucleotide sequence encoding an epitope between the first MCS and the selectable marker. First and second homology arms can also be prepared. Each of the first and second homology arms can include a polynucleotide sequence that is homologous to the 5′ and 3′ regions flanking the target gene locus, respectively. The first and second homology arms can be cloned into the first and second MCSs, respectively.

In yet another aspect, the targeting vector can include a recombinant adenoassociated virus (AAV) virion. The recombinant AAV virion can include first and second inverted terminal repeats (ITRs) linked to the first and second MCSs of the modification cassette, respectively. The selectable marker can include a promoter linked to a polynucleotide encoding resistance to an antibiotic and the epitope can include three tandem arrayed FLAG epitopes.

The present invention also relates to a method for characterizing an endogenous polypeptide of a somatic cell. The method includes knocking-in an epitope tag-encoding polynucleotide into an endogenous locus of a somatic cell so that an epitope tagged endogenous polypeptide is expressed by the cell. The tagged polypeptide can be immunoprecipitated with an antibody that is specific to the tag. The immunoprecipitated polypeptide can then be characterized.

In an aspect of the invention, the epitope tagged endogenous polynucleotide can be knocked-in by homologous recombination mediated knock-in. The homologous recombination mediated knock-in can be performed by transfecting the somatic cell with a targeting vector. The targeting vector can include a delivery vehicle linked to a modification cassette. The modification cassette can include the epitope tag-encoding polynucleotide.

In a further aspect, the targeting vector can be constructed to genetically modify an endogenous target gene locus in the somatic cell. The modification cassette can further include first and second multiple cloning sites (MCSs) and a polynucleotide sequence encoding a selectable marker conferring drug resistance. The targeting vector can also be packaged for delivery to the somatic cell.

In another aspect, the targeting vector can be constructed by ligating the delivery vehicle with the modification cassette and inserting the polynucleotide sequence encoding an epitope between the first MCS and the selectable marker. First and second homology arms can also be prepared. Each of the first and second homology arms can include a polynucleotide sequence that is homologous to the 5′ and 3′ regions flanking the target gene locus, respectively. The first and second homology arms can be cloned into the first and second MCSs, respectively.

In yet another aspect, the targeting vector can include a recombinant adenoassociated virus (AAV) virion. The recombinant AAV virion can include first and second inverted terminal repeats (ITRs) linked to the first and second MCSs of the modification cassette, respectively. The selectable marker can include a promoter linked to a polynucleotide encoding resistance to an antibiotic and the epitope can include three tandem arrayed FLAG epitopes.

The present invention further relates to a targeting vector for genetically modifying an endogenous target gene locus in a somatic cell. The targeting vector includes a delivery vehicle and a modification cassette linked to the delivery vehicle. The modification cassette includes a polynucleotide sequences encoding an epitope. The modification cassette can be integrated into the target gene locus by homologous recombination without affecting endogenous transcriptional regulation of the target gene locus.

In an aspect of the invention, the targeting vector includes a recombinant AAV virion. The modification cassette further includes first and second MCSs and a polynucleotide sequence encoding a selectable marker conferring drug resistance. The recombinant AAV virion can include first and second ITRs linked to the first and second MCSs of the modification cassette, respectively. The first and second MCSs can have first and second homology arms respectively inserted therein. Each of the first and second homology arms can include a polynucleotide sequence that is homologous to the 5′ and 3′ regions flanking the target gene locus, respectively. The selectable marker can also include a promoter linked to a polynucleotide encoding resistance to an antibiotic. The epitope can include three tandem arrayed FLAG epitopes.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a schematic diagram of tagging endogenous protein with 3xFLAG. (a) rAAV-Neo-Lox P-3xFLAG vector. ITR: AAV inverted terminal repeats; MCS: multiple cloning site; CMV: Cytomegalovirus promoter; Neo: Neomycin resistance gene. (b) Diagram of knock-in strategy. (c) Western Blots of genomic PCR of parental (P) and STAT3 3xFLAG KI cells (clone 1 and 2). Arrow indicates the targeted allele. (d) Western Blots of genomic PCR of the MRE11 loci in RKO and LOVO CRC cells. (e) and (f) Western Blots of genomic PCR of PTPN14 locus and a novel gene (N gene) locus.

FIG. 2 illustrates 3xFLAG tagged proteins can be utilized for Western blot and immunoprecipitation. (a). Western blots of parental and STAT3 3xFLAG KI DLD1 CRC cells with indicated antibodies. (b). Cell lysates of STAT3 3xFLAG KI cells were immunoprecipitated with anti-FLAG antibody and Western blotting was performed with anti-STAT3 antibody. (c). Western blots of MRE11 KI RKO and LOVO CRC cells with indicated antibodies. (d). Immunoprecipitation analyses of the MRE11 KI RKO and LOVO CRC cells. (e) and (f). Western blot and immunoprecipitation analyses of the PTPN14 and Novel gene (N protein). (g) Western blot analysis on equal amounts lysates of parental cells (P), STAT3, PTPN14, N gene and MRE11 3xFLAG KI cells with indicated antibodies.

FIG. 3 illustrates 3xFLAG tagged proteins can be utilized for immunofluorescent staining. Parental and STAT3 3xFLAG KI cells were treated with or without IL-6 for 30 min and fixed. Immunofluorescent staining was performed with a rabbit anti-STAT3 antibody and a mouse monoclonal anti-FLAG antibody (Sigma M2).

FIG. 4 illustrates ChIP analysis of wild-type and FLAG-tagged STAT3 in DLD1 cells. (a) ChIP-Western analysis of STAT3 in FLAG-tagged and parental cells. The lane indicated by the dash corresponds to a wild-type DLD1 lysate that was not subjected to IP. (b) Histogram of signal ratios of FLAG-STAT3 chromatin-immunoprecipitated DNA versus random-sheared total genomic DNA. The distinct tail at the right-hand end corresponds to DNA fragments enriched by FLAG-STAT3 ChIP (see inset). Tiled oligos that displayed the top 0.25% ratios are located to the right of the red bar. (c) STAT3 binding profiles from a 500 kb region on chromosome 21. Normalized raw ratio data from the indicated ChIP-chip experiments are plotted. The top 0.25% is displayed as a dotted horizontal line. An expanded view of a positive signal from the left is shown on the right.

FIG. 5 illustrates comparison of sites bound by wild-type and FLAG-tagged STAT3. (a) The maximum signal intensity ratios for each STAT3-occupied site is plotted on the x and y axes and correspond to the filled circles (n=214). For comparison, 15 randomly selected regions are plotted as open circles. (b) Signal intensities of STAT3 bound regions are plotted as a heatmap. Note the high degree of overlap of STAT3-bound sites found in all 3 experiments. For comparison, 15 randomly selected sites are included (bracket).

FIG. 6 illustrates the enrichment of selected STAT3 binding sites. Real-time ChIP-PCR analysis of 18 randomly selected sites determined by ChIP-chip to be bound by FLAG-STAT3 in tagged lines at high confidence. Fold enrichment is relative to FLAG-ChIP in wild-type cells. Chromosome coordinates of each amplicon are shown in the Legend.

FIG. 7 illustrates ChIP-chip profiles of wildtype CHD7 and single allele FLAG-tagged CHD7. (a) DNA from a CHD7-ChIP in wildtype DLD1 cells (top), and 2 FLAG-ChIP's in CHD7-FLAG-tagged DLD1 cells (middle and bottom) were hybridized to Agilent arrays containing oligos spanning several ENCODE regions. The ˜400 Kb genomic interval shown corresponds to an ENCODE region on human chromosome 7. Normalized raw ratio data from the indicted experiments are plotted. Note the similarity in the profiles of wildtype CHD7 and two independently derived clones in which one allele of the CHD7 gene was tagged. (b) Expanded view of 3 positive signals from a.

FIG. 8 illustrates the diagram of one-step USER cloning of rAAV-3xFLAG knock-in vector construction including SEQ ID NO:7 in cassette A, SEQ ID NO:8 in cassette B, and SEQ ID NOs:3-6 in (b).

FIG. 9 illustrates a flow diagram of the overview of the rAVV-mediated tagging approach and the standard approach for production of polyclonal antibodies.

### DETAILED DESCRIPTION

The present invention relates generally to a method for genetically modifying somatic cells, and more particularly to a method for modifying an endogenous gene or chromosomal locus to characterize endogenous polynucleotides and endogenous polypeptides. The present invention is at least partially based on the discovery that a recombinant adenoassociated virus (rAAV) can be used to “knock in” epitope tag sequences into targeted gene loci in somatic cells by homologous recombination, and that tagged endogenous proteins can be expressed and exploited for various immunoassays, such as Western blot, immunoprecipitation, immunofluorescence and ChIP-chip analyses. The present invention therefore provides a method for characterizing an epitope-tagged polypeptide, a method for producing an epitope-tagged endogenous polypeptide, a method of characterizing and/or identifying endogenous polynucleotide and polypeptide interactions, a targeting vector for genetically modifying an endogenous target gene locus in a somatic cell, and a related method for preparing the targeting vector.

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the present invention.

In the context of the present invention, the term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, all “mimetic” and “peptidomimetic” polypeptide forms, and retro-inversion peptides (also referred to as all-D-retro or retro-enantio peptides).

As used herein, the term “polynucleotide” refers to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, siRNAs, microRNAs, and ribonucleoproteins. The term also encompasses nucleic acids, i.e., oligonucleotides, containing known analogues of natural nucleotides, as well as nucleic acid-like structures with synthetic backbones.

As used herein, the term “antibody” refers to whole antibodies, e.g., of any isotype (IgG, IgA, IgM, IgE, etc) and includes fragments thereof which are also specifically reactive with a target polypeptide. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain polypeptide. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term “antibody” also includes polyclonal, monoclonal, or other purified preparations of antibodies, recombinant antibodies, monovalent antibodies, and multivalent antibodies. Antibodies may be humanized and may further include engineered complexes that comprise antibody-derived binding sites, such as diabodies and triabodies.

As used herein, the term “subject” refers to any warm-blooded organism including, but not limited to, human beings, pigs, rats, mice, dogs, goats, sheep, horses, monkeys, apes, rabbits, cattle, etc.

As used herein, the term “epitope” refers to a portion of a molecule, such as protein that is recognized by the immune system. An epitope can include, but is not limited to, an amino acid, a polynucleotide, a carbohydrate, a protein, a lipid, a capsid protein, a coat protein, a polysaccharide, a sugar, a lipopolysaccharide, a glycolipid, a glycoprotein, and/or part of a cell or a biological entity, such as a virus particle. It will be appreciated that the term can be used interchangeably with other terms such as “antigen,” “paratope binding site,” “antigenic determinant,” and/or “determinant.”

As used herein, the term “delivery vehicle” refers to any genetic element, such as a plasmid, phage, transposon, cosmid, chromosome, virus, virion, etc., which is capable of replication when associated with the proper control elements and which can transfer gene sequences between cells. Delivery vehicles can include cloning and expression vehicles, as well as viral vectors.

As used herein, the term “modification cassette” refers to a stretch of polynucleotides, typically linear, which contain any combination of DNA sequences, for example, including promoters, regulatory elements, coding sequences, polyadenylation sequences, splice acceptor/splice donor sequences, epitope sequences, etc., that can modify a target gene locus once it is inserted into it. Such insertion can occur by any means including, but not limited to, homologous recombination.

As used herein, the term “targeting vector” refers to a polynucleotide construct that contains sequences homologous to endogenous chromosomal polynucleotide sequences flanking a target gene locus. The flanking homology sequences, referred to as “homology arms”, direct the targeting vector to a specific chromosomal location within the genome by virtue of the homology that exists between the homology arms and the corresponding endogenous sequence, and introduce the desired genetic modification by homologous recombination.

As used herein, the terms “homology” or “homologous” refer to the percent similarity between two polynucleotides or two polypeptide moieties. Two polynucleotide, or two polypeptide sequences are “substantially homologous” to each other when the sequences exhibit at least about 50% to about 99% or more, for example, sequence similarity or sequence identity over a defined length of the molecules. The term “substantially homologous” can also refer to sequences showing complete identity to a specified polynucleotide or polypeptide sequence.

As used herein, the term “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can also be used to aid in the analysis of similarity and identity.

As used herein, the term “recombinant virus” refers to a virus that has been genetically altered, e.g., by the addition or insertion of a heterologous polynucleotide construct into the particle.

As used herein, the term “AAV virion” refers to a complete virus particle, such as a wild-type (wt) AAV virus particle (comprising a linear, single-stranded AAV polynucleotide genome associated with an AAV capsid protein coat). In this regard, single-stranded AAV polynucleotides of either complementary sense, e.g., “sense” or “antisense” strands can be packaged into any one AAV virion while maintaining the infectivity of both strands are equally.

As used herein, the term “recombinant AAV virion” or “rAAV virion” refers to an infectious, replication-defective virus including an AAV protein shell, encapsidating a heterologous polynucleotide sequence of interest which is flanked on both sides by AAV inverted terminal repeats (ITRs). A rAAV virion can be produced in a suitable host cell which has had an AAV vector, AAV helper functions, and/or accessory functions introduced therein. In this manner, the host cell can be rendered capable of encoding AAV polypeptides that are required for packaging the AAV vector (containing a recombinant polynucleotide of interest) into infectious recombinant virion particles for subsequent gene delivery.

As used herein, the term “transfection” refers to the uptake of foreign DNA by a cell, and a cell has been “transfected” when exogenous DNA has been introduced inside the cell membrane. A number of transfection techniques are generally known in the art and can be used to introduce one or more exogenous DNA moieties, such as a modification cassette into suitable host cells.

As used herein, the term “linked” refers to an arrangement of genetic elements wherein the elements configured so as to perform their usual function. For example, a control sequence can be linked to a coding sequence capable of affecting the expression of the coding sequence.

As used herein, the term “homologous recombination” refers to the process of DNA recombination based on sequence homology. The term embraces both crossing over and gene conversion.

The present invention provides a targeting vector and related method for tagging endogenous polypeptides or proteins with an epitope to facilitate characterization of the polypeptides and proteins as well as polynucleotides the polypeptides and proteins interact with. As described in more detail below, the present invention provides several advantages over transgenic expression of recombinant proteins. The epitope-tag sequences are knocked into endogenous gene loci by homologous recombination so that transcriptional regulation by native promoters and enhancers is maintained. The present invention also obviates the need for cloning tagged full-length cDNAs, which can be particularly challenging for large transcripts. Moreover, the present invention can be used to tag one or multiple alleles at a given locus, allowing for analysis of genes with aberrant copy numbers. Additionally, the epitope tag can serve as a universal epitope for multiple applications so that detection methods can be standardized.

The method for characterizing an endogenous polynucleotide, an endogenous polypeptide, endogenous polypeptide-polynucleotide interactions (e.g., transcription factor-DNA interactions) can include, for example, introducing epitope tag-encoding DNA into an endogenous target gene locus of a somatic cell by homologous recombination mediated knock-in so that an epitope tagged endogenous polypeptide is expressed by the cell. The epitope tag-encoding DNA can be introduced in to the cell by transfecting the somatic cell with a targeting vector capable of genetically modifying an endogenous target gene locus in a somatic cell. The targeting vector can comprise a delivery vehicle linked to a modification cassette. The delivery vehicle can include any genetic element, such as a plasmid, phage, transposon, cosmid, chromosome, virus, virion, etc., which is capable of replication and which can transfer the modification cassette between cells. The delivery vehicle can include cloning and expression vehicles, as well as viral vectors. Viral vectors can include any of the obligate intracellular parasites having no protein-synthesizing or energy-generating mechanism. Viral vectors can include RNA or DNA genomes surrounded by a lipid bilayer and a coating structure composed of proteins. Examples of viral vectors useful in the practice of the present invention can include, but are not limited to, baculoviridiae, parvoviridiae, picornoviridiae, herpesviridiae, poxviridae, adenoviridiae and picotinaviridiae.

In an example of the present invention, the delivery vehicle can include an AAV vector. The AAV vector can be derived from an AAV serotype including, without limitation, AAV-1, AAV-2, AAV-3, AAV-4, AAV-5, AAV-6, AAV-7 and/or AAV-8. The AAV vector can have one or more of the AAV wild-type genes deleted in whole or part, such as the rep and/or cap genes, but retain functional flanking ITR sequences. Functional ITR sequences are necessary for the rescue, replication, and packaging of the AAV vector. The AAV vector can include at least those sequences required in cis for replication and packaging (e.g., functional ITRs) of the virus. The ITRs need not be the wild-type polynucleotide sequences, and may be altered, e.g., by the insertion, deletion or substitution of polynucleotides, as long as the sequences provide for functional rescue, replication and packaging.

The modification cassette can be linked to the delivery vehicle and can comprise a stretch of polynucleotides capable of modifying a target gene locus. The modification cassette can be inserted into a target gene locus via homologous recombination, for example, without affecting endogenous transcriptional regulation of the target gene locus. More particularly, the modification cassette can include a polynucleotide sequence encoding an epitope. The epitope can comprise a portion of a molecule, such as polypeptide, that is recognized by the immune system (e.g., an antibody). The epitope can include, but is not limited to, an amino acid, a nucleotide, a carbohydrate, a protein, a lipid, a capsid protein, a coat protein, a polysaccharide, a sugar, a lipopolysaccharide, a glycolipid, a glycoprotein, and/or part of a cell or of a biological entity, such as a virus particle. Examples of epitopes which may be included as part of the modification cassette can include, but are not limited to, the FLAG epitope, the c-myc epitope, the hemagglutinin epitope, the green fluorescent protein (GFP) epitope, the histadine epitope, and the glutathione-s-transferase epitope. The FLAG epitope, for example, is a synthetic epitope that consists of eight amino acid residues (SEQ ID NO: 1). Additionally, the c-myc epitope is derived from the human c-myc gene and contains 10 amino acid residues (SEQ ID NO: 2).

The modification cassette can include other genetic elements or components to facilitate integration of the modification cassette at a target gene locus. For example, the modification cassette can include a polynucleotide encoding a selectable marker. The selectable marker can allow for isolation of transfected cells expressing the marker in a population of cells. Examples of selectable markers can include, but are not limited to, neomycin phosphotransferase (neo), hygromycin phosphotransferase (hygro), puromycin-N-acetyl-transferase (puro), and/or markers that provide other types of selection cues, such as LacZ or fluorescing proteins such as GFP.

The selectable marker can also include negative selection genes such as Herpes Simplex Virus thymidine kinase (HSV-tk) and fusions of HSV-tk with neo, hygro or puro, or other selectable marker genes known in the art. The selectable marker may or may not be under the transcriptional control of an exogenous promoter, such as PGK, human ubiquitin C promoter, or cytomegalovirus (CMV) promoter. Additionally or optionally, selection marker can be flanked by sites recognized by recombinases. For example, a selectable marker comprising a neo gene can be flanked by Lox P sites.

The modification cassette can additionally include at least one multiple cloning site (MCS). The MCS can comprise a relatively short sequence of DNA which contains a number of closely-spaced recognition sequences for restriction endonucleases. The MCS can facilitate introduction of a desired polynucleotide sequence or sequences into the modification cassette. For example, the MCS can facilitate insertion of at least one homology into the modification cassette. The homology arm can comprise a polynucleotide sequence substantially homologous to a region flanking the target gene locus. The homology arm can direct the targeting vector to a specific chromosomal location within the genome by virtue of the homology that exists between the homology arm and the corresponding endogenous sequence. Such homology can facilitate introduction of a desired genetic modification at a target gene locus by homologous recombination, for example.

It will be appreciated that other polynucleotide sequences capable of turning on, turning off, enhancing, down-regulating, or otherwise modulating expression of all or a portion of the modification cassette may be included as part of the modification cassette. For example, polyadenylation sequences (which function as transcriptional termination signal sequences) can also be included as part of the modification cassette.

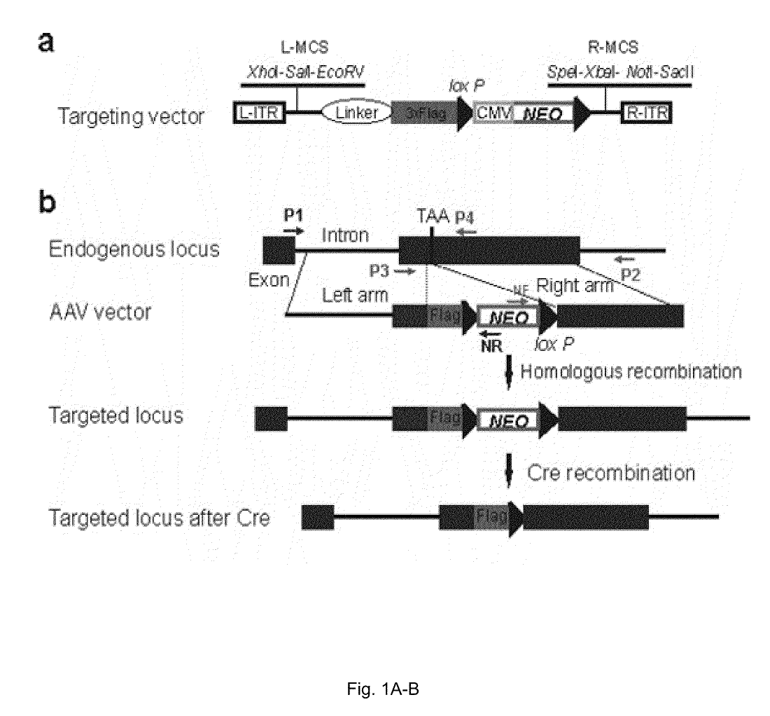

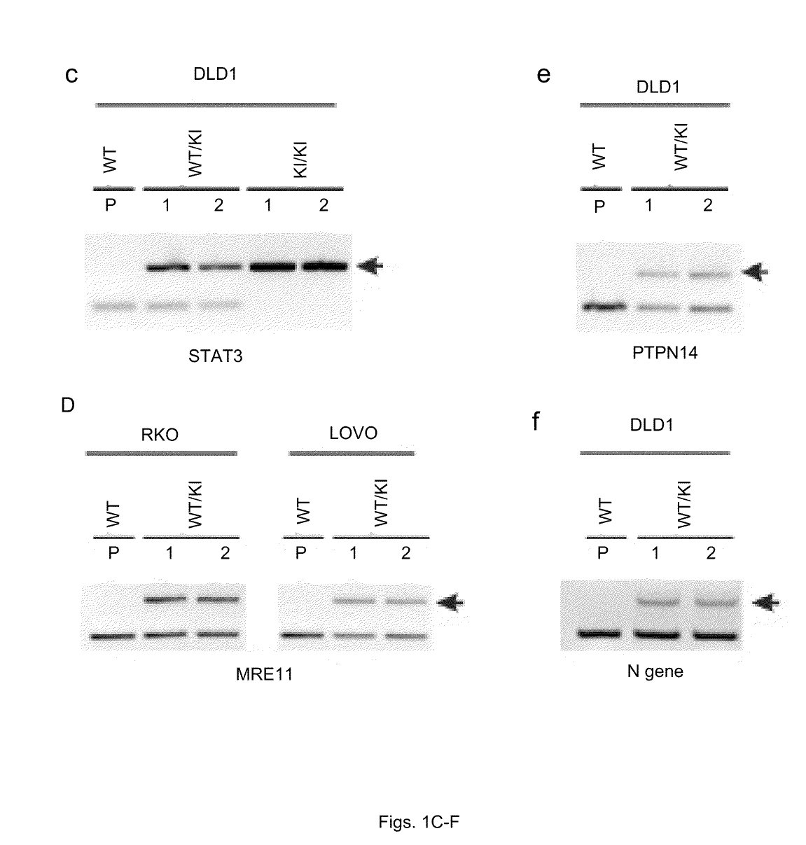

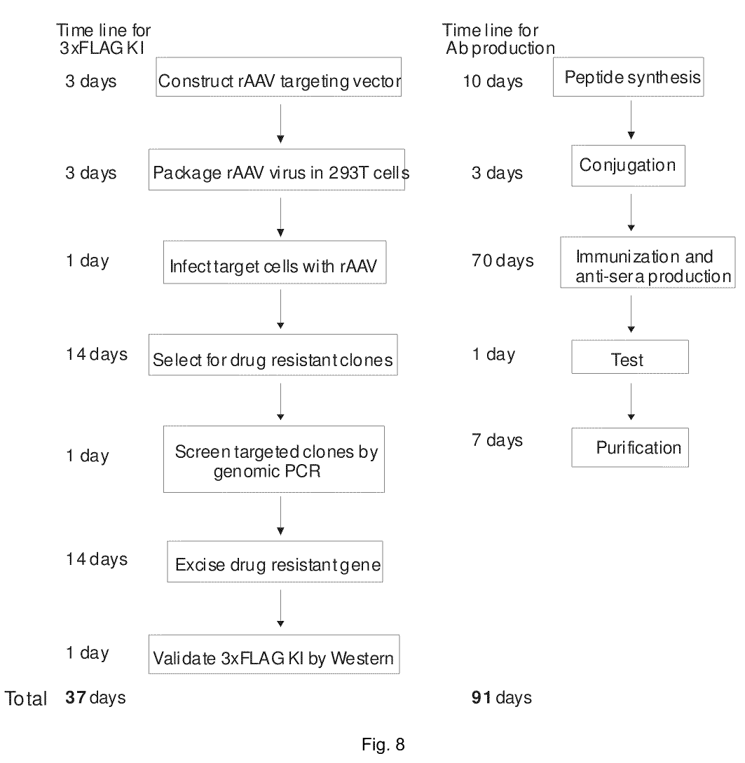

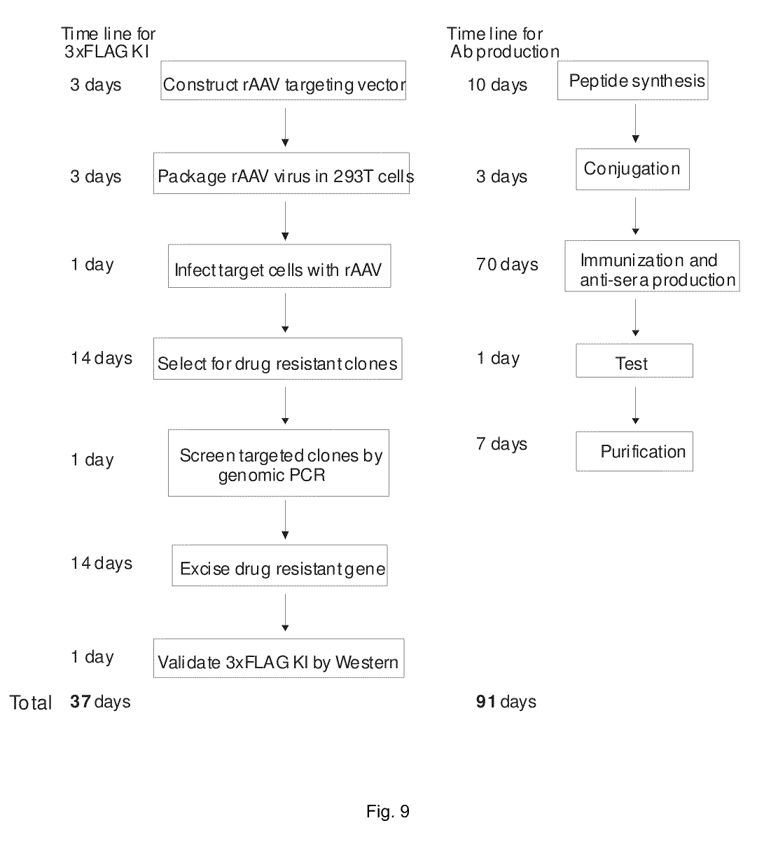

In an example of the present invention, the targeting vector can have a structure as shown in FIGS. 1A and 9B. As shown in FIGS. 1A and 8B, the delivery vehicle of the targeting vector can comprise an AAV virion. The AAV virion can include left and right ITRs, a bacterial replication origin (not shown), and an ampicillin resistance gene (not shown). The modification cassette can include first and second MCSs which are respectively linked to the left and right ITRs. FIG. 1A shows the first and second MCSs (“Cassette A” and “Cassette B”, respectively) in detail. Additionally, the modification cassette can include three tandem arrayed FLAG epitopes (3xFLAG) and a polynucleotide encoding neo. A first FLAG epitope can be linked to the first MCS, and a third FLAG epitope can be linked to the neo marker. The neo marker can be linked to the second MCS and can be under the control of a CMV promoter (FIG. 1A). As shown in FIG. 1A, the neo marker can be flanked by Lox P sites.

In another example of the present invention, the targeting vector can have a structure as shown in FIG. 8B. As shown in FIG. 9B, for example, the delivery vehicle of the targeting vector can comprise an AAV virion. The AAV virion can include left and right ITRs. The modification cassette can include left and right homology arms, as well as three tandem arrayed FLAG epitopes and a polynucleotide encoding neo. As shown in FIG. 8B, the left homology arm can be disposed between the left ITR and a FLAG epitope, and the right homology arm can be disposed between the right ITR and the neo marker. Each of the left and right homology arms can comprise a polynucleotide sequence that is substantially homologous to the 5′ and 3′ regions flanking the target gene locus, respectively.

The targeting vector and the components contained therein can be constructed using standard molecular biology techniques well known to the skilled artisan (see, e.g., Sambrook, J., E. F. Fritsch and T. Maniatis. Molecular Cloning: A Laboratory Manual, Second Edition, Vols. 1, 2, and 3, 1989; Current Protocols in Molecular Biology, Eds. Ausubel et al., Greene Publ. Assoc., Wiley Interscience, NY). For example, methods for producing rAAV virions are disclosed in U.S. Patent Pub. Nos. 2006/0292123 A1, 2007/0172949 A9, and 2003/0157688 A1, and U.S. Pat. No. 7,241,447.

Prior to constructing the targeting vector, a desired target gene locus can be selected for genetic modification. The target gene locus can be in any organelle of a somatic cell, including the nucleus and mitochondria, and can be an intact gene, an exon, an intron, a regulatory sequence, any region between genes, or a combination thereof. A variety of approaches can be used for selecting the target gene locus for genetic modification. For example, the selection approach can be based on specific criteria, such as detailed structural or functional data, or it can be selected in the absence of such detailed information as potential genes or gene fragments are predicted through the various genome sequencing projects. It should be noted that it is not necessary to know the complete sequence of the target gene locus to apply the methods of the present invention.

By way of example, the targeting vector can be constructed by ligating a delivery vehicle (e.g., an AAV vector having left and right ITRs) with a modification cassette. The modification cassette can include, for example, a polynucleotide fragment containing a neo resistance gene cassette flanked by two Lox P sites, and left and right MCSs. Next, a polynucleotide sequence encoding an epitope, such as a polynucleotide sequence encoding three tandem arrayed FLAG epitopes can be amplified using PCR. The PCR products can be digested with a restriction endonuclease, such as ECO R1, and then inserted into the modification cassette. For a given target gene locus, first and second homology arms can be prepared by PCR amplification of the 5′ and 3′ regions flanking the target gene locus. The PCR product corresponding to the 5′ flanking region can then be cloned in-frame with the 3xFLAG sequence, and the PCR product corresponding to the 3′ flanking region can be cloned in-frame with the neo resistance gene.

FIG. 8B illustrates another example of a method for constructing the targeting vector. In FIG. 9B, the uracil-specific excision reagent (USER) cloning technique can be used to facilitate assembly of multiple DNA fragments in a single reaction by in vitro homologous recombination and single-strand annealing. In this system, the targeting vector can include a modification cassette with two inversely oriented nicking endonuclease sites separated by restriction endonuclease site(s). The targeting vector can be digested and nicked with restriction endonulceases, yielding a linearized vector with non-complimentary, single-stranded overhangs of about 8 polynucleotides.

PCR amplification can be used to generate homology arms for cloning into the modification cassette. Using PCR, a single deoxyuridine (dU) residue can be placed about 8 nucleotides from the 5′-end of each PCR primer. In addition to the dU, the PCR primers can contain a sequence compatible with each unique overhang on the modification cassette. After amplification, the dU can be excised from the PCR products which are flanked by 3′ single-stranded extensions of about 8 polynucleotides and are complementary to the modification cassette overhangs. When mixed together, the linearized modification cassette and PCR products can directionally assemble into a recombinant molecule through the complementary single-stranded extensions.

To make the targeting vector compatible with the USER cloning system, a first homologous arm (Cassette A) and a second homologous arm (Cassette B) can be respectively inserted between the left ITR and 3xFLAG sequences, and between the right Lox P site and right ITR of the AAV-oriented nicking endonuclease sites (Nt.BbvCl) (which are separated by restriction endonuclease sites, Xbal). After treatment with Nt.BbvCl and Xbal restriction enzymes, the AAV-USER-3xFLAG construct can be digested into a 3xFLAG-Lox P-Neo-Lox P construct flanked by two 5′ single-stranded overhangs and a vector backbone flanked by two 5′ overhangs. PCR can then be used to amplify left and right homologous arms from genomic DNA. For example, the polynucleotide sequence GGGAAAGU (SEQ ID NO: 3) can be added to the 5′ end of the forward left arm primers, and the polynucleotide sequence GGAGACAU (SEQ ID NO: 4) can be added to the reverse left arm primers. Additionally, the polynucleotide sequence GGTCCCAU (SEQ ID NO: 5) can be added to the forward right arm primers, and the polynucleotide sequence GGCATAGU (SEQ ID NO: 6) can be added to the reverse left arm primers. The PCR products can be treated with 1 U of USER enzyme (New England Biolabs, Ipswich, Mass.) to generate single-stranded overhangs. Finally, the left and right homologous arms can be mixed with the two vector fragments and then followed by bacterial transformation.

In another aspect of the present invention, an epitope-tagged polypeptide can be produced. The epitope-tagged polypeptide can be produced by constructing the targeting vector (as described above), and then packaging the targeting vector for delivery to a somatic cell. In an example of the present invention, the targeting vector shown in FIG. 1A can be packaged by mixing about 2.5 μg of the targeting vector with pAAV-RC and pHelper plasmids (about 2.5 μg of each) from the AAV Helper-Free System (Stratagene, Cedar Creek, Tex.). The targeting vector can then be transfected into HEK 293 cells using LIPOFECTAMINE (Invitrogen Corp., Carlsbad, Calif.).

Next, the targeting vector can be dissolved in Opti-MEM reduced-serum media (Invitrogen Corp., Carlsbad, Calif.) to a total volume of about 750 μl (i.e., if the volume of DNA is about 50 μl, then the volume of Opti-MEM can be about 700 μl). Similarly, about 54 μl of LIPOFECTAMINE can be dissolved in Opti-MEM to a total volume of about 750 μl. The volumes can be combined and the DNA-LIPOFECTAMINE mix incubated at about room temperature for about 15 minutes. HEK 293T cells at about 70-80% confluence in a 75 cm2 flask can then be washed with Hanks' Balanced Salt Solution (HBSS) (HyClone, Logan, Utah) and about 7.5 ml of Opti-MEM added to the flask. To this, the DNA-LIPOFECTAMINE mixture can be added dropwise and the cells incubated at about 37° C. for about 3-4 hours. The Opti-MEM can be replaced with 293 growth medium and the cells allowed to grow for about 72 hours prior to harvesting virus.

The virus can be harvested according to the AAV Helper-Free System instructions, for example, with minor modifications. Briefly, the media can be aspirated from the flask and the 293 cells scraped into about 1 ml of phosphate-buffered saline (PBS) (Invitrogen Corp., Carlsbad, Calif.), transferred to a microfuge tube of about 2 ml, and then subjected to about three cycles of freeze-thaw. Each cycle can consist of about a 10 minute freeze in a dry ice-ethanol bath, and about a 10 minute thaw in about 37° C. water bath (followed by vortexing after each thaw). The lysate can then be clarified by centrifugation at about 12 000 r.p.m. in a microfuge to remove cell debris. The supernatant containing virus can then be divided into three aliquots of about 330 μl each and frozen at about −80° C. The viral preparation can include about 3×108 viral particles/ml.

After packaging the targeting vector, at least one packaged targeting vector can be introduced into a somatic cell (e.g., a mammalian somatic cell) using standard methodologies, such as transfection mediated by calcium phosphate, lipids or electroporation. The cells in which the modification cassette has been introduced successfully can be selected by exposure to any number of selection agents, depending on the selectable marker that has been engineered into the modification cassette. For example, if the selectable marker is the neo gene, then cells that have taken up the modification cassette can be selected in G418-containing media. Cells that have not taken up the modification cassette will die, whereas cells that have taken up the modification cassette will survive.

Cells which have been genetically modified (i.e., the modification cassette has been integrated at the target gene locus) can be identified using a variety of approaches and assays. Such assays can include, but are not limited to: (a) quantitative PCR (Lie et al., Curr Opin Biotechnol, 9:43-8, 1998); (b) quantitative assays using molecular beacons (Tan et al., Chemistry 6:1107-11, 2000); (c) fluorescence in situ hybridization or FISH (Laan et al., Hum Genet 96:275-80, 1995) or comparative genomic hybridization (CGH) (Forozan et al., Trends Genet, 13:405-9, 1997); (d) isothermic DNA amplification (Lizardi et al., Nat Genet, 19:225-32, 1998); and (e) quantitative hybridization to an immobilized probe(s) (Southern, J. Mol. Biol. 98: 503, 1975).

In an example of the present invention, HEK 293T cells can be grown in 25 cm2 flasks and infected with packaged virus when about 75% confluent. At the time of infection, medium can be aspirated and about 4 ml of medium containing about 50-250 μl of viral lysate (about 0.5-2.5×105 viral particles) can be added to each flask. The cells can be washed with PBS and then detached with trypsin (Invitrogen Corp., Carlsbad, Calif.) about 24 hours after infection. The cells can then be re-plated in eight 96-well plates in medium containing geneticin (Invitrogen Corp., Carlsbad, Calif.) at a final concentration of about 1 mg/ml. Drug-resistant colonies can be grown for about 3-4 weeks. At the end of the selection period, genomic DNA can be extracted from single clones growing in 96-well plates using the Lyse-N-Go reagent (Pierce Biotechnology, Inc., Rockford, Ill.). Locus-specific integration can be assessed by PCR using a primer that anneals outside the homology region and another that anneals with neo, for example.

After confirming locus-specific integration of the modification cassette, the selectable marker can be excised from the modification cassette. To remove the neo marker from cells having the modification cassette, for example, the cells can be infected with an adenovirus that expresses Cre recombinase (see, e.g., Kohli, M. et al., Nucleic Acids Res 32, e3, 2004). Briefly, the cells can be plated at limited dilution in a non-selective medium about 24 hours after infection. After about two weeks, single cell clones can be plated in duplicate and about 0.4 mg/ml of geneticin added to the cell cultures. The cells can then be cultured and assessed for the presence of epitope-tagged polypeptides, as described in more detail below.

In another aspect of the present invention, an immunoassay can be performed to detect the presence of an epitope-tagged endogenous polypeptide. An immunoassay can generally include any biochemical test capable of measuring the concentration of a substance in a biological liquid (e.g., serum, urine, cell culture media, etc.) using the reaction of an antibody to its antigen. Examples of immunoassays can include, but are not limited to, Western blots, Northern blots, Southern blots, immunohistochemistry, chromatin immunoprecipitation (ChIP) assays (including ChIP-chip assays), ELISA, e.g., amplified ELISA, radioimmunoassay, immunoprecipitation, immunofluorescence, flow cytometry, immunocytochemistry, and the like.

Depending upon the results of the immunoassay, for example, the epitope-tagged polypeptide can be characterized in a variety of ways. Examples of how epitope-tagged polypeptides can be characterized are provided below and can include, but are not limited to:

(1) subcellular localization of tagged proteins (e.g., immunofluorescence analysis of tagged protein(s) in permeabilized cells; ultrastructural analysis of tagged protein(s) in cells with gold-conjugated tag-specific antibodies and electron microscopy; and Western blot analysis of tagged full-length and truncated protein(s) in cell membrane subfractions);

(2) determination of protein-protein interactions (e.g., immunoprecipitation of tagged protein(s) from cell extract and gel analysis of precipitate; and immobilization of tagged protein(s) on Protein A-agarose to study in vitro assembly of a multiprotein complex);

(3) functional assay of tagged protein(s) (e.g., immunoprecipitation of tagged protein(s) from cell extract and activity assay, such as phosphorylation of immunoprecipitate; and Western blot detection of tagged protein(s) in cellular extracts under varying conditions, such as activation or suppression of a cell function);

(4) tracking movement of tagged protein(s) within a cell (e.g., immunoprecipitation of tagged protein(s) from cell extract after pulse-chase labeling of cellular protein(s); immunofluorescence analysis of tagged protein(s) in intact cell membranes; localization of tagged protein(s) in cells with gold-conjugated tag-specific antibody and electron microscopy; and localization of tagged protein(s) in cells with confocal immunofluorescence microscopy); and

(5) characterization of new proteins (e.g., Western blot analysis of tagged protein(s) expressed by transfected cell lines; purification of tagged protein(s) from cell extract by affinity chromatography; and immunoprecipitation of tagged protein(s) from cell extract and gel analysis of subunit structure).

In another example of the present invention, a ChIP assay can be performed to characterize and/or identify interactions or binding of an endogenous polypeptide, such as a transcription factor that has been tagged with a 3xFLAG epitope, with chromatin DNA. Methods for performing ChIP assays are well known in the art. Generally, in ChIP assay, DNA-protein complexes in cells may be effectively fixed in place by cross-liking with formaldehyde. The DNA-protein complex can then be fragmented into small pieces, and an antibody directed against an epitope tag (i.e., 3xFLAG) can be used to precipitate the DNA-protein complex. The cross-linking can then be reversed, and the identity and quantity of the DNA can be determined by PCR. Alternatively, when one wants to find where the protein or polypeptide binds on a genome-wide scale, a DNA microarray can be used (e.g., ChIP-on-chip or ChIP-chip) allowing for the characterization of the cistrome.

The following examples are for the purpose of illustration only, and are not intended to limit the scope of the claims, which are appended hereto.

### EXAMPLE

### Cells and Reagents

The human colorectal cancer cell lines DLD1, RKO and LOVO were grown in McCoy's 5A modified medium (Invitrogen) supplemented with 10% fetal bovine serum (HyClone) and penicillin/streptomycin (Invitrogen). Antibodies: mouse anti-FLAG monoclonal antibody (Sigma); rabbit anti-STAT3 antibody (Santa Cruz); mouse anti-MRE11 monoclonal antibody (Novus).

### AAV Titration Assay

The rAAV was titrated by real-time PCR as described by Veldwijk, M. R. et al., Mol Ther 6:272-278 (2002). Briefly, 10 μl of rAAV stock was mixed with 10 μl of salmon sperm DNA (1 mg/ml) and 20 μl of 2 M NaOH. The mixture was then inoculated at 56° C. for 30 min and then neutralized by adding 19 μl of 2 M HCl. The rAAV lysates were diluted 10 fold and 1 μl of dilutant was mixed with 2 μl of 5 μM forward primer (5′-tgaatgaactgcaggacgag-3′), 2 μl of 5 μM reverse primer (5′-caatagcagccagtcccttc-3′) and 12.5 μl of SYBR green PCR mix in a total volume of 25 μl. To calculate the copy number, the rAAV-Neo targeting vector was serially diluted in the range of 10³ to 10⁶ copies per μl as the real-time PCR standards.

### Gene Targeting and Isolation of Recombinant Cell Lines

Cells (DLD1, RKO and LOVO) were grown in 25 cm² flasks and infected with rAAV when 75% confluent (˜3×10⁶). At the time of infection, medium was aspirated and 4 ml of medium containing 50-250 μl of rAAV lysate (0.2-1×10⁸ viral particles) was added to each flask. Cells were washed with PBS buffer and detached with trypsin (Invitrogen) 24 hours after infection. Cells were replated in 96-well plates in medium containing geneticin (Invitrogen) at a final concentration of 1 mg/ml. drug resistant colonies were grown for 10-14 days (˜3,000 G418 resistance clones/T25 flask). At the end of the selection period, genomic DNA was extracted from single clones growing in 96-well plates using the Lyse-N-Go reagent (Pierce). Locus-specific integration was assessed by PCR using a primer that annealed outside the homology region and another that annealed with neo. Positive clones were confirmed by PCR across both homology arms.

### Cre-Mediated Excision of the Drug Resistance Marker in Targeted Cells

To remove the drug resistance marker from correctly targeted clones, cells were infected with an adenovirus that expresses the Cre recombinase, as described by Kohli, M. et al., Nucleic Acids Res 32, e3 (2004). Cells were plated at limiting dilution in non-selective medium, 24 hours after infection. After 2 weeks, single cell clones were plated in duplicate and 0.4 mg/ml geneticin was added to one set of wells. After 1 week of growth, clones that were geneticin-sensitive were expanded for further analysis.

### Western Blot and Immunoprecipitation Analysis

Cells were lysed in RIPA buffer with protease inhibitors and phosphatase inhibitors (50 mM Tris-HCl, pH 8.0, 0.5% triton X-100, 0.25% sodium deoxycholate, 150 mM sodium chloride, 1 mM EDTA, 1 mM sodium orthovnadate, 50 mM NaF, 80 μM β-glyerophosphate, and 20 mM sodium pyrophosphate). Western blots and immunoprecipitation were performed essentially as described by Zhang, X. et al., PNAS 104:4060-4064 (2007).

### Immunofluorescent Staining

CRC cells were seeded on glass cover slips, grown to 50% confluence, and fixed with 4% paraformaldehyde for 30 minutes at room temperature. The fixed cells were permeabilized with 0.2% Triton X 100 at room temperature for 5 minutes and then blocked with IMAGE-IT FX signal enhancer (Invitrogen) at room temperature for 30 minutes. Immunofluorescent staining was performed with indicated primary and secondary antibodies (Invitrogen). Nuclei were stained with DAP1 (1 μg/ml) at room temperature for 20 minutes. Images were captured with a Zeiss LSM 510 laser scanning confocal microscope.

### Chromatin Immunoprecipitation Coupled with DNA Microarray (ChIP-Chip Analysis)

The protocol described here was adapted from previously published studies (Scacheri, P. C. et al., PLoS Genet 2, e51(2006)). Briefly, for each ChIP-chip experiment, 1 to 2×10⁸ IL-6 stimulated cells were cross-linked with 1% formaldehyde for 15 minutes at room temperature, harvested, and rinsed with 1×PBS. Cell nuclei were isolated, pelleted, and sonicated. DNA fragments were enriched by immunoprecipitation with antibodies directed against either FLAG (Sigma M5, etc.) or STAT3 (sc-482, Santa Cruz). After heat reversal of the cross-links, the enriched DNA was amplified by ligation-mediated PCR (LM-PCR) and then fluorescently labeled with Cy5 dUTP (Amersham Biosciences, Piscataway, N.J.). A sample of DNA that was not enriched by immunoprecipitation was subjected to LM-PCR and labeled with Cy3 dUTP. ChIP-enriched and unenriched (input) labeled samples were cohybridized to ENCODE microarrays (NimbleGen, Madison, Wis.). All ChIP-chip experiments were performed in biological triplicate.

### Analysis of ChIP Tiling Array Data

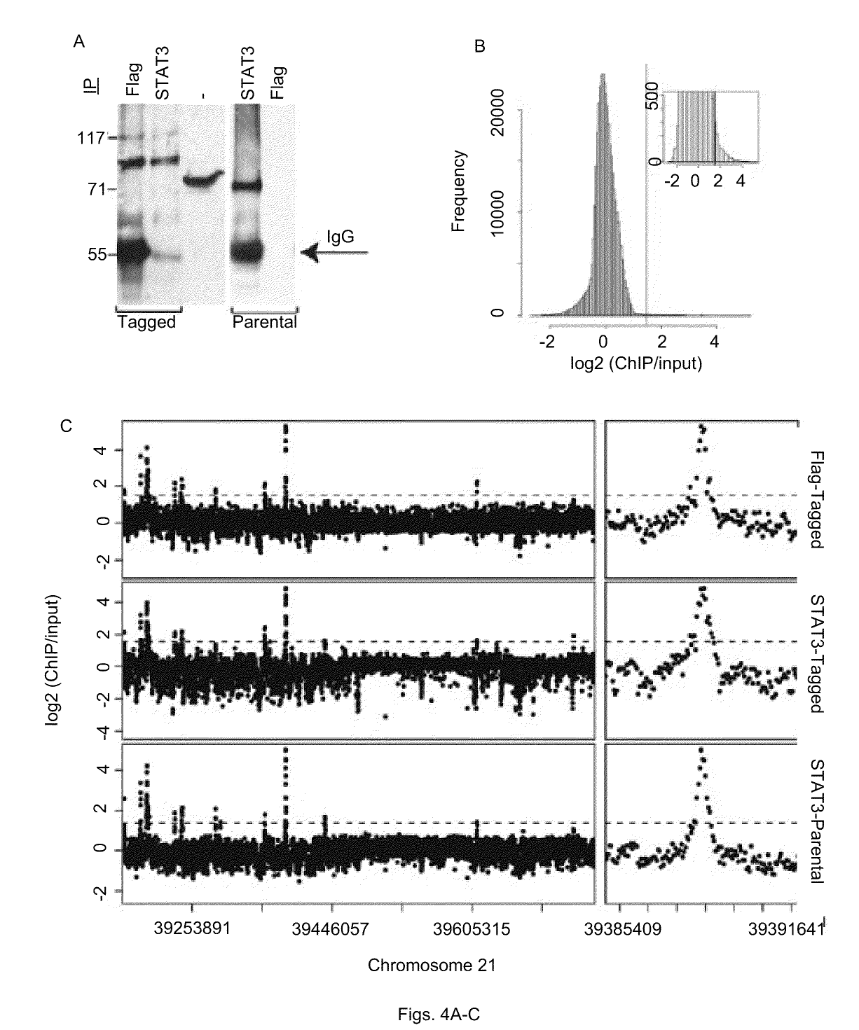

Raw array data were normalized using bi-weight mean using the NimbleScan Version 2.1 software (NimbleGen Systems, Inc.). Log2 ratios (cy5/cy3) from biological replicates were averaged. These ratios were used to perform a Chi-square test on sliding 500-bp windows to identify regions with a higher than expected number of oligos in the top 0.25% on the log-ratio distribution (indicated by the red bar in FIG. 4B and the dotted line in FIG. 4C).

### Real-Time ChIP-PCR

Standard ChIP with FLAG and STAT3 antibodies was performed on both FLAG-tagged and untagged DLD1 cells. PCR primers were designed to amplify 170-200-bp fragments from 18 genomic regions determined by ChIP-chip to be enriched for FLAG-tagged STAT3 in DLD1 cells at P<1×10⁻¹⁵. Two non-enriched (non-target) regions on chromosome 1 were also amplified for comparison. Real-time PCRs were carried out in duplicate on each chromatin immunprecipitated and input DNA sample using SYBR green PCR mix in an Applied Biosystems 7500 Real-Time PCr machine (Foster City, Calif.). To account for differences in DNA quantity, for every genomic region studied, a DCt value was calculated for each sample by subtracting the Ct value for chromatin immunoprecipitated sample from the Ct value obtained for the input. Raising 2 to the DCt power yielded the relative amount of PCR product. Average values for the 18 target regions were compared to the average values for the two non-target regions. Data were then normalized to ChIP-PCR results from FLAG-ChIP experiments in wild-type (untagged) DLD1 cells.

### Correlation of STAT3 Peaks to the Annotated Genome

Using the Table Browser function in the UCSC Genome Browser, 179 STAT3 peaks identified by FLAG ChIP-chip analysis of tagged STAT3 were compared to the selected annotations based on the HG17 genome assembly. Data were compared to 10 sets of 179 random sequences within the ENCODE regions. To ensure accuracy, randomly generated sequences were matched in length to those identified by FLAG-ChIP-chip. For the comparison to conserved regions, we utilized consensus elements generated by the ENCODE Multi-Species Analysis group. These elements were generated from nine different combinations of three conservation algorithms (phastCons, binCons, and GERP) and three sequence alignment methods (TBA, MLAGAN, and MAVID) applied to the ENCORE region sequences of 28 vertebrate species as defined in the September 2005 ENCODE MSA sequence freeze and the MSA species guide tree. Three different stringencies were used. The loose set of constrained sequences represent bases identified as being constrained by any conservation algorithm on any alignment. The moderate set of constrained sequences is derived from bases shown to be constrained by at least two of the three conservation algorithms on at least two of the three alignments. Finally, the strict set of constrained sequences represent only those bases that were constrained using all three conservation programs on all three multiple-sequence alignments. A z-test for two proportions was used to determine if differences between the FLAG-ChIP and random datasets were statistically significant. P values were corrected for multiple testing.

### Motif Identification

Both tagged and untagged ChIP-chip hits were tested for enrichment of any motifs that correspond to known transcription factors. We scanned each of the 517 binding matrices corresponding to vertebrate motifs in the TRANSFAC database. The database is redundant so that many motifs have several slightly different binding matrices. To compute enrichment, we used the Clover algorithm, with two different sets of background sequences. For the first set, we randomly shuffled the input sequence set while maintaining the dinucleotide composition. The second set was the union of all ChIP-chip hits generated by the ENCODE Transcription Regulation Group at the 5% false discovery rate cut off. Motifs with significant P-values (<0.01) for both background sequence sets were reported. Similar matrices were grouped to circumvent the redundancy of TRANSFEC, as well as the inherent limitation of our matrix-centric analysis, i.e., if a motif has similar matrix to another motif that is truly enriched, the former would also appear to be enriched. Two matrices were considered similar if the score computed with the Malign program was greater than 0.03.

### Construction of a Universal Targeting Vector for rAAV-Mediated Knock-in of an Epitope Tag

To generate a universal targeting vector for tagging endogenous proteins, we constructed a rAAV-3xFLAG-Neolox P vector (FIG. 1a) which included the following elements: (1) Left (L)- and Right (R)-multiple cloning sites (MCS) for inserting sequences that are homologous to target loci; (2) 3xFLAG sequences; (3) a neomycin resistance gene flanked by two lox P sites; and (4) internal terminal repeat (ITR) sequences, which are required for packaging the targeting plasmid into virus.

rAAV targeting vectors are constructed by insertion of left homologous arm (˜1 kb genomic DNA sequences upstream of the stop codon of the target gene) and right homologous arm (˜1 kb genomic DNA sequences downstream of the stop codon of the target gene) into the rAAV-Neo-Lox P-3xFLAG vector. Targeting rAAV viruses are then packaged in 293T cells. Cells are infected with the targeting virus and selected for geneticin-resistant clones. To identify correctly targeted clones, the clones are then screened by genomic PCR with primers complementary to sequences in the neomycin resistance gene and upstream of the left homologous arm (indicated as P1 and NR). Confirmative genomic PCR is also performed on positive clones using primers complementary to the neomycin resistance gene and to a sequence downstream of the right homologous arm (indicated as NF and P2) To excise the neomycin gene cassette, the targeted clones are infected with adenovirus expressing Cre-recombinase and limit diluted into 96-well plate. Genomic PCRs to amplify 100-200 bp fragments surrounding the Lox P insertion site (primers are indicated as P3 and P4) are used for identifying clones with the neomycin cassette excised.

### Packaging of rAAV Targeting Constructs

The targeting construct made above (2.5 μg) was mixed with pAAV-RC and pHelper plasmids (2.5 μg of each) from the AAV Helper-Free System (Stratagene) and transfected into HEK 293T cells (ATCC) using LIPOFECTAMINE (Invitrogen). The DNA was dissolved in Opti-MEM reduced-serum media (Invitrogen) to a total volume of 750 μl (i.e., if volume of DNA was 50 μl, volume of Opti-MEM was 700 μl). Similarly, 54 μl of LIPOFECTAMINE was dissolved in Opti-MEM to a total volume of 750 μl. The two tubes were combined and the DNA-LIPOFECTAMINE mix was incubated at room temperature for 15 min. HEK 293T cells at 70-80% confluence in a 75 cm² flask were washed with Hanks' Balanced Salt Solution (HBSS, HyClone) and then 7.5 ml Opti-MEM was added. To this, the 1.5 ml DNA-LIPOFECTAMINE mixture was added dropwise, and the cells were incubated at 37° C. for 3-4 hours. The Opti-MEM was replaced with 293 growth medium and the cells were allowed to grow for 72 hours prior to harvesting virus. Virus was harvested according to the AAV Helper-Free System instructions with minor modifications. Briefly, the media was aspirated from the flask and the 293 cells were scraped into 1 ml of phosphate-buffered saline (Invitrogen), transferred to a 2 ml microfuge tube, and subjected to three cycles of freeze-thaw. Each cycle consisted of 10 min freeze in a dry ice-ethanol bath, and 10 min thaw in a 37° C. water bath, vortexing after each thaw. The lysate was then clarified by centrifugation at 12 000 r.p.m. in a microfuge to remove cell debris and the supernatant containing rAAV was divided into three aliquots of ˜330 μl each and frozen at −80° C. The rAAV preparation generally contained ˜3×10⁸ genome particles/ml.

### Successful Targeting of Multiple Loci in Human Colorectal Cancer (CRC) Cells

We engineered the targeting vector described above to knock in epitope-tag sequences into the 3-prime end of five autosomal genes: STAT3 (signal transducer and activator of transcription 3), PTPN14 (protein tyrosine phosphatase nonreceptor 14), MRE11 (meiotic recombination 11), CHD7 (chromodomain helicase DNA-binding protein 7), and N-gene, which encodes a novel protein. For tagging of STAT3, PTPN14, CHD7, and N-gene, DLD1 colorectal cancer cells (CRC) were infected with the recombinant targeting viruses. RKO and LOVO cells (also colorectal cells) were infected for rAAV-mediated tagging of MRE11. With the exception of N-gene, which is duplicated in the human genome, all other 3 genes are present in normal copy number. G418 resistant clones were screened for homologous recombination by genomic PCR. The targeting frequency ranged from 1-2%. To excise the neomycin resistance gene, targeted clones were infected with adenoviruses expressing Crerecombinase (FIG. 1b). To select clones with successful deletion of the drug selection marker, genomic PCR was performed to amplify a diagnostic genomic fragment containing the inserted Lox P and 3xFLAG sequences. As shown in FIGS. 1C-F, the PCR products of the targeted alleles (indicated by the arrows) are larger than that of the wild-type alleles, consistent with the acquisition of the 3xFLAG and LoxP sequences (CHD7 data not shown). Two independently derived heterozygous STAT3 3xFLAG KI clones were re-infected with targeting virus to generate homozygous 3xFLAG KI cells (FIG. 1c). The data indicate that rAAV-mediated targeting approach is generally applicable to multiple loci in CRC cell lines.

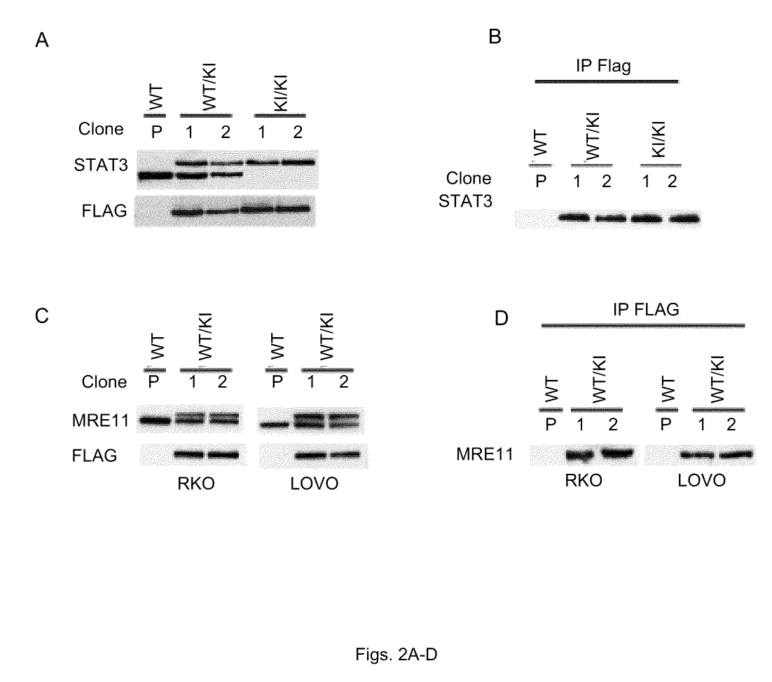

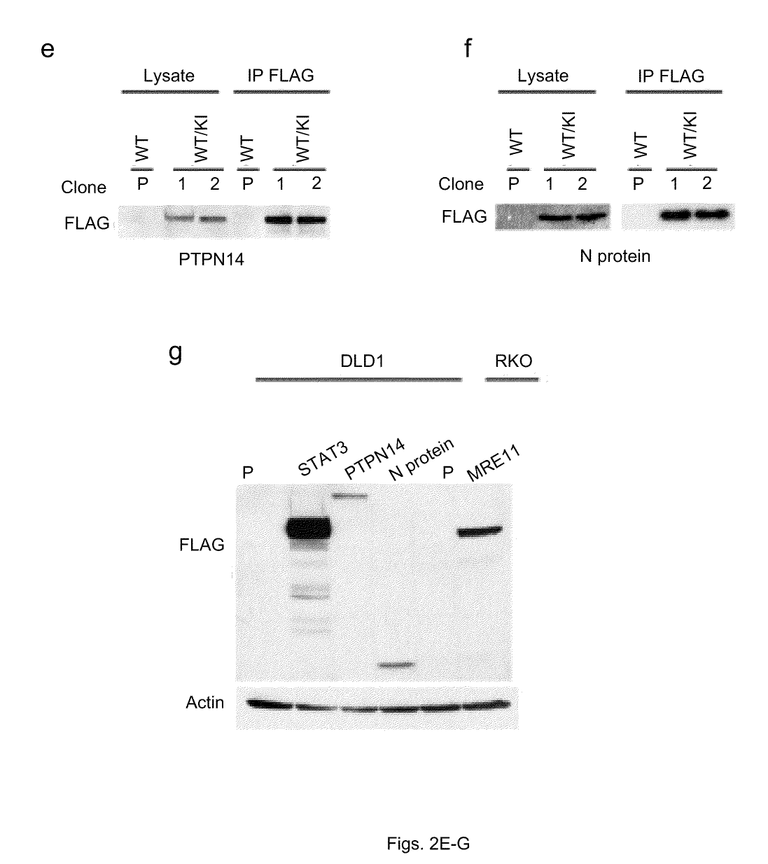

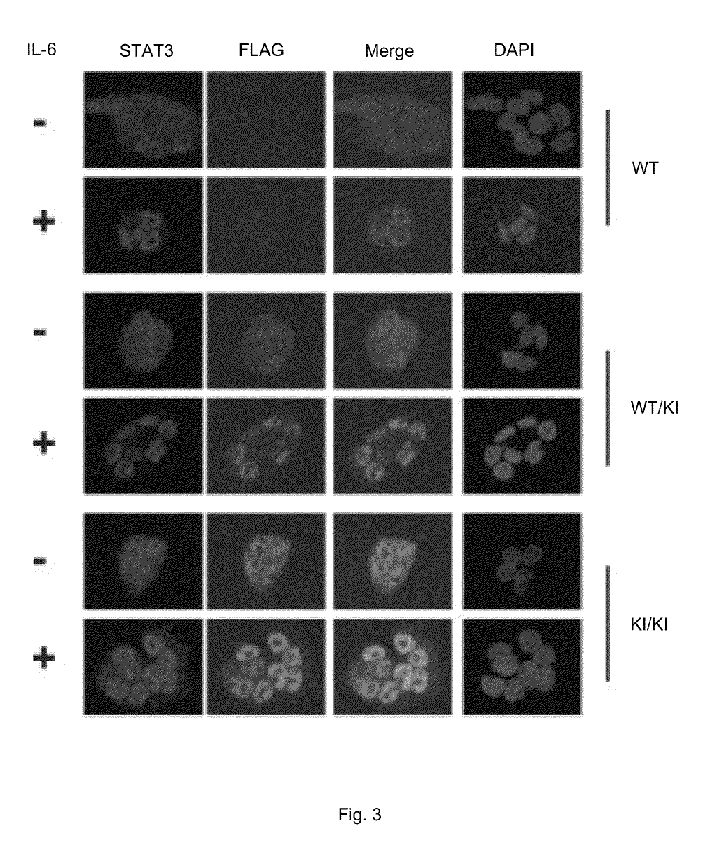

We performed Western and IP analysis of endogenous FLAG-tagged proteins. As shown in FIG. 2, all epitope tagged proteins were readily detectable by Western blot and IP with anti-FLAG antibodies (CHD7 data not shown). Furthermore, the wild-type and tagged forms in heterozygous cells were nearly identical in quantity, suggesting that the presence of the tags does not alter expression levels. Interestingly, with only one of the four alleles of N gene tagged with FLAG epitope, the N protein was successfully detected by western blot and immunoprecipitation analyses (FIG. 2f). It is also worth noting that the targeting approach works for proteins of either high (STAT3) or low (PTPN14) abundance (FIG. 2g). To determine whether the FLAG tag can be exploited for immunofluorescence, we co-stained the 3xFLAG STAT3 KI cells with a rabbit anti-STAT3 polyclonal antibody and a mouse anti-FLAG monoclonal antibody. As shown in FIG. 3, STAT3 and FLAG staining were co-localized, indicating that the immunostaining with anti-FLAG antibody was specific for STAT3 proteins. Moreover, FLAG-tagged STAT3 translocated into the nucleus following stimulation with interleukin-6 (IL-6), suggesting that addition of the 3xFLAG tag does not disturb STAT3 function.

### ChIP-Chip Analysis of FLAG-Tagged and Wild-Type STAT3

For an epitope tag to be suitable for ChIP, (1) an available antibody must be capable of efficiently immunoprecipitating the tagged protein within the context of chromatin, (2) the antibody must be highly specific for the tagged protein, and (3) the tag must not alter the factor's genomic distribution. To address these issues, we performed chromatin immunoprecipitation on (1) FLAG-tagged STAT3 with FLAG antibodies, (2) FLAG-tagged STAT3 with STAT3 antibodies, and (3) STAT3 in wild-type DLD1 cells using STAT3 antibodies. All cells were stimulated with IL-6 prior to cross-linking. Western blot analyses indicated that both FLAG and STAT3 antibodies were capable of cleanly immunoprecipitating wild-type and tagged STAT3 starting from a fragmented chromatin cell fraction (FIG. 4a). Chromatin immunoprecipitated DNA and input DNA were amplified and co-hybridized to tiled microarrays that span the ENCODE regions, corresponding to a representative 1% of the human genome. Raw data were normalized, and the mean intensity ratios of oligonucleotide probes from each of three biological replicate experiments were plotted as a histogram (FIG. 4b and data not shown). Mean intensity ratios were also plotted by their position along each chromosome. Representative examples of the data from an ENCODE region are shown in FIG. 4c, where putative STAT3 binding sites are represented by multiple clusters of neighboring probes with signal intensities that stand out above background. A computer program incorporating a sliding window and threshold approach, ACME (Algorithm for Capturing Microarray enrichment) was used to identify genomic sites enriched for STAT3 binding at high confidence (P<1×10⁻¹⁵). Within the ENCODE regions, we identified 179 binding sites using FLAG antibodies in the tagged DLD1 cells, 153 binding sites using STAT3 antibodies in tagged DLD1 cells, and 161 binding sites using STAT3 antibodies in wild-type DLD1 cells. Using the Clover algorithm and all motifs in the TRANSFAC database, we tested ChIP-chip hits for enrichment of motifs that correspond to known transcription factor binding sites. As expected, the STAT3 motif was significantly enriched in ChIP-chip hits from both tagged and untagged cells (data not shown).

### Overlap of STAT3 Binding Sites

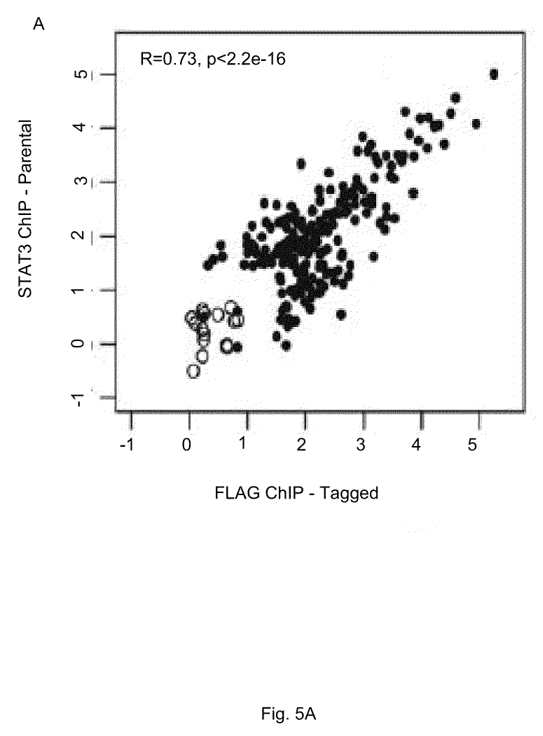

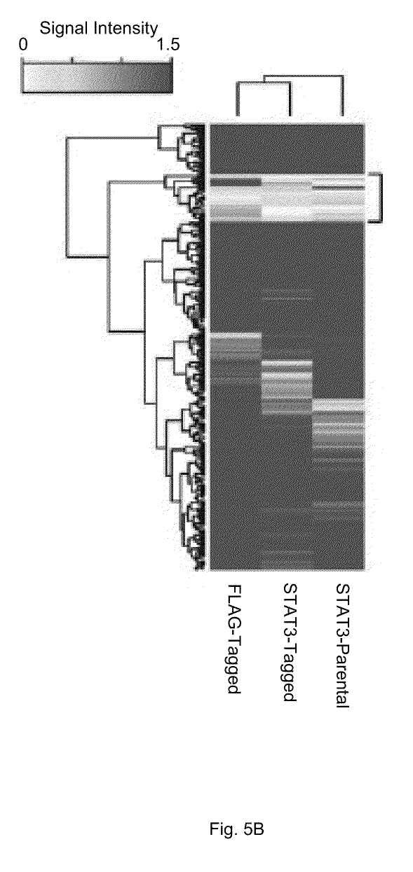

The binding profiles from FLAG and STAT3 ChIP-chip experiments appear strikingly similar for the 0.5 MB ENCODE region shown in FIG. 4c. To systematically determine the overlap of STAT3 binding sites for the remaining 29.5 MB within the ENCODE regions, we selected all sites that were identified by ChIP-chip with antibodies to FLAG or wild-type STAT3 (n=214), and plotted the maximum mean signal intensity value for each site in a scatter plot (FIG. 5a) and heatmap (FIG. 5b). The plots reveal excellent correlations between sites identified using FLAG antibodies and those found with STAT3 antibodies, suggesting that the vast majority of binding sites identified between experiments overlap. Some of the nonoverlapping sites could be due to differences in antibody sensitivity, subtle variations in growth conditions, or experimental variability. However, we think that most of the variation is the result of threshold issues related to processing the raw data with the ACME algorithm, and not true false negatives. This is supported by both the heatmap in FIG. 5b and by visualization examination of the raw data. Regardless of the minor differences, the data suggest that the FLAG antibodies are specific for STAT3, and the presence of the tag does not significantly alter the genomic distribution of STAT3.

### Validation of STAT3 Binding Sites

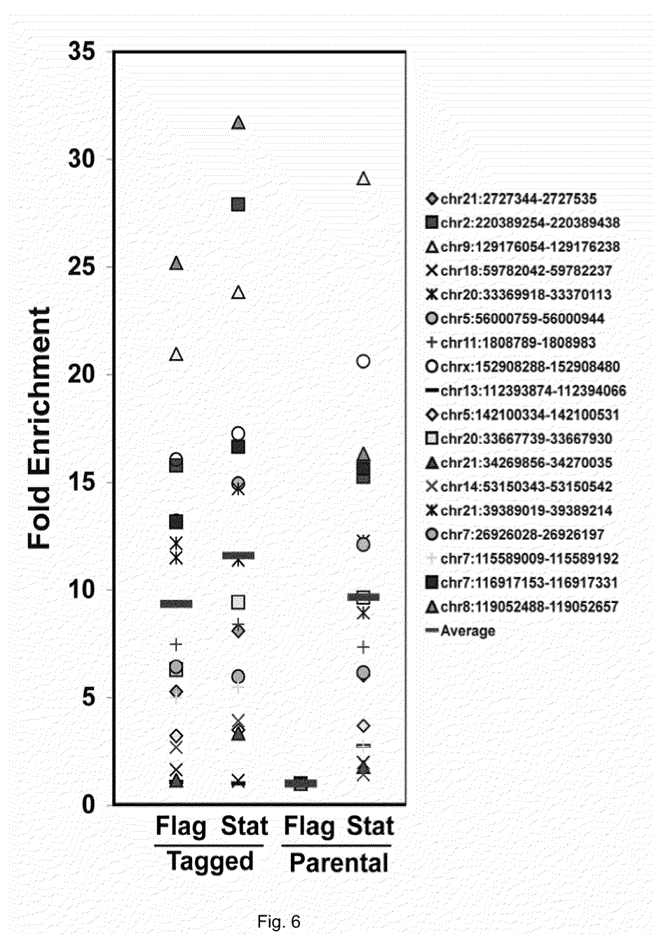

We used a standard approach that combines conventional ChIP and real-time PCR to assess the reliability of our ChIP-chip data. We arbitrarily selected 18 regions that were found enriched for STAT3 binding in FLAG ChIP-chip experiments from tagged DLD1 cells. These regions were tested for enrichment in chromatin immunoprecipitated material from experiments performed using either FLAG or STAT3 antibodies in tagged and wild-type cells. As expected, no enrichment was detected in FLAG ChIP experiments from untagged cells. In ChIP-PCR from wild-type STAT3 cells, 15/18 (83%) sites were confirmed by real-time PCR to be enriched >2-fold over FLAG-ChIP in wild-type cells (FIG. 6). Moreover, the relative amounts of enrichment for each site tested were similar between the wild-type and tagged cell lines, indicating that the efficiency of ChIP was independent of the cell line and antibody. The data not only validate the reliability of the ChIP-chip method for detecting STAT3 binding sites, but also support the ChIP-chip findings indicating that the sites identified with FLAG antibodies accurately represent STAT3 binding sites.

### ChIP-Chip Analysis of Single-Allele FLAG-Tagged CHD7

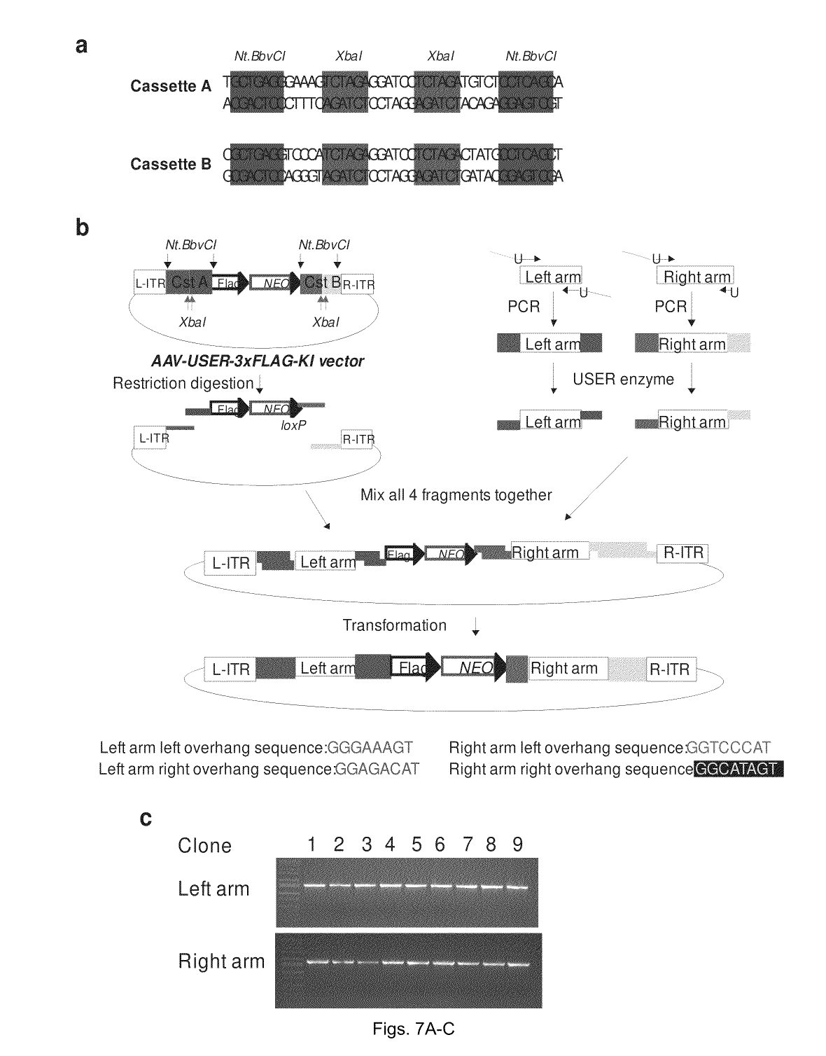

For the FLAG-ChIP-chip experiments described above, both alleles of the STAT3 gene were tagged by rAVV-mediated knockin. To determine if one tagged allele is sufficient for ChIP, we performed ChIP-chip analyses of CHD7, for which only one allele was tagged with 3xFLAG. It is noteworthy that the abundance of CHD7 is very low in comparison to STAT3, and the size of CHD7 (336 KDa) is much larger than STAT3 (92 kDa). As indicated in FIG. 7, the binding profiles of wild-type CHD7 and FLAG-tagged CHD7 are nearly identical. These data suggest that tagging only a single copy of a given gene is sufficient for global ChIP analysis. The data also suggest that low abundance, high-molecular weight proteins are amenable to the tagging/ChIP approach.

### Implementation of a High-Throughput Cloning Method for Construction of Multiple Targeting Vectors

Currently, the rAAV targeting vectors are constructed by sequential insertion of left and right homologous arms into the rAAV targeting vector backbone through restriction enzyme cutting and re-ligation. At best, this process takes 10-14 days. The desired characteristics of high-throughput targeting-vector construction are to be able to insert the left and right homologous arms in a sequence-independent manner and preferably in a single step.

Recently, homologous recombination-based cloning methods have been successfully exploited for highthroughput vector construction. The New England Biolabs has developed the USER (uracil-specific excision reagent) cloning technique, which facilitates assembly of multiple DNA fragments in a single reaction by in vitro homologous recombination and single-strand annealing. In this system, the vector contains a cassette with two inversely oriented nicking endonuclease sites separated by restriction endonuclease site(s). The vector is then digested and nicked with restriction endonucleases, yielding a linearized vector with 8-nucletide single-stranded, non-complimentary overhangs. To generate target molecules for cloning into this vector, a single deoxyuridine (dU) residue is placed 6-10 nucleotides from the 5′-end of each PCR primer. In addition to the dU, the PCR primers contain sequence that is compatible with each unique overhand on the vector. After amplification, the dU is excised from the PCR products with a uracil DNA glycosylase and an endonuclease (the USER enzyme), generating PCR products flanked by 3-prime, 8 are complimentary to the vector overhangs. When mixed together, the linearized vector and PCR products directionally assemble into a recombinant molecule through complementary single-stranded extensions.

To make the rAAV-mediated targeting vector compatible with the USER cloning system, we inserted cassette A (Cst A) between LITR and 3xFLAG sequences, and cassette B (Cst B) between the right lox P site and RITR of the AAV-3xFLAG knockin vector to generate the AAV-USER-3xFLAG-KI vector (FIG. 8). These cassettes contain two inversely oriented nicking endonuclease sites (Nt. BbvCI) separated by restriction endonuclease sites (XbaI). After treatment with Nt.BbvCI and XbaI restriction enzymes, the AAV-USER-3xFLAG-KI vector is digested into a Tag-lox P-Neo-lox P fragment flanked by two 5′ singlestranded overhangs (blue and red sticks) and a vector backbone flanked by two 5′ overhangs (green and yellow sticks). PCR is then used to amplify left and right homologous arms from genomic DNA. The sequence GGGAAAGU (SEQ ID NO: 3) is added to the 5′ of the forward left arm primers, and GGAGACAU (SEQ ID NO: 4) is added to the reverse left arm primers. GGTCCCAU (SEQ ID NO: 5) is added to the forward right arm primers and GGCATAGU (SEQ ID NO: 6) to the reverse left arm primers. The PCR products are then treated with the USER enzymes to generate single-stranded overhangs. Finally, the left and right arms are mixed with the two vector fragments followed by bacterial transformation. We used this approach to construct a β-catenin 3xFLAG targeting vector. Colony PCRs were performed to screen for insertion of the left and right homologous arms. All of the 9 randomly picked bacterial colonies harbored the AAV targeting plasmids with both arms inserted (FIG. 8C). Restriction maps and sequencing data confirmed that all the plasmids were assembled in the correct orientation. We also used this strategy to construct 6 AAV knockout vectors. The cloning efficiency for 5 of them was 100% and the remaining one was 80%. Clones were verified error-free by sequence analysis.

The newly developed 3xFLAG AAV knockin vector cloning method has several advantages over the standard approach: (1) extremely fast. It takes only one step in two days with the new method, instead of the traditional two-step approach which requires at least 10 days. (2) Highly efficient. For all six genes tested thus far, the cloning efficiency ranged from 80-100%. (3) Very simple. The USER cloning eliminates ligation and restriction digestion of the inserted fragments. The restriction digested vector fragments can be prepared in a single batch for multiple uses. (4) Generally applicable. Unlike restriction digestion and re-ligation, the new method is not dependent on the presence of restriction sites within the sequences to be inserted. One protocol should work for construction of multiple, different constructs. The only variable is the design of PCR primers for targeted homologous arms. Therefore, the approach is readily adaptable for high-throughput vector construction.

### Comparison of the 3xFLAG rAAV KI Method to Standard Polyclonal Antibody Production

FIG. 9 depicts an overview of the current targeting method (left), and the method required for antibody production (right). Although multiple steps are required in both cases, the rAAV-tagging procedure is more than 2 times faster than polyclonal antibody production, and this is without considering the time required for testing the antibodies for their use in multiple applications, e.g., ChIP. The inherent limitation of the rAAV-tagging approach is that different cell lines of interest must be tagged independently; once a good ChIP-capable antibody is generated, it can be readily utilized in multiple cell types. However, the rAAV-mediated approach is more likely to yield a product that is suitable for ChIP. In addition, multiple cell lines can be tagged in parallel. Lastly, the tagging approach can be done at a fraction of the cost required for antibody production ($100 per cell line versus $1000 for polyclonal antibody production).

In summary, we have (1) developed a method for targeted knock-in of epitope sequences encoding 3xFLAG; (2) demonstrated successful targeting of five loci in human colorectal cancer cells; and (3) shown that the 3xFLAG tagged proteins can be exploited for multiple applications including Western blot, Immunoprecipitation, Immunofluorescence, and ChIP-chip. With respect to the application of ChIP-chip, we have shown that presence of the tag on one allele is sufficient for analysis, and that the genomic distribution of the tagged proteins is similar to that of corresponding wild type proteins. Lastly, using the recently developed USER cloning method, we have significantly simplified the process of targeting vector construction.

From the above description of the invention, those skilled in the art will perceive improvements, changes and modifications. For example, the present invention can be utilized for purposes other than the immunoassays described herein. For instance, the present invention could be used to distinguish protein isoforms generated from alternatively spliced transcripts or to introduce tandem affinity purification tags and thereby provide opportunities for purifying protein complexes (e.g., for crystallography). The present invention can also be used for introducing polynucleotide sequences other than epitope tags. For example, Lox P sequences could be introduced for targeted deletion of genomic regions, including those that harbor regulatory elements, miRNAs, or ultraconserved regions. Additionally, the present invention could be used to engineer cell lines and/or transgenic animals with new mutations. Such improvements, changes and modifications are within the skill of the art and are intended to be covered by the appended claims. All publications, patents, and patent applications cited in the present application are herein incorporated by reference in their entirety.

Having described the invention, the following is claimed:

1. A method of determining polynucleotide binding sites of an endogenous polypeptide of a somatic cell, the method comprising:
knocking-in an epitope tag-encoding polynucleotide into an endogenous locus of the somatic cell so that an epitope tagged endogenous polypeptide is expressed by the cell and binds to the endogenous polynucleotide;
immunoprecipitating the tagged polypeptide and the endogenous polynucleotide with an antibody that is specific to the tag; and
determining the identity of the immunoprecipitated polynucleotide.

2. The method of claim 1, the polynucleotide comprising DNA of a genome of the somatic cell.

3. The method of claim 1, the polypeptide comprising a transcription factor.

4. The method of claim 1, the identity of the immunoprecipitated polynucleotide being determined using at least one of a polynucleotide microarray or PCR.

5. The method of claim 1, the epitope tagged endogenous polynucleotide being knocked-in by homologous recombination mediated knock-in.

6. The method of claim 1, the epitope tag-encoding polynucleotide being knocked-in by transfecting the somatic cell with a targeting vector, the targeting vector including a delivery vehicle linked to a modification cassette, the modification cassette including the epitope tag-encoding polynucleotide.

7. The method of claim 6, the step of transfecting a somatic cell with a targeting vector further comprising the steps of:
constructing the targeting vector to genetically modify an endogenous target gene locus in the somatic cell, the modification cassette further including first and second multiple cloning sites (MCSs) and a polynucleotide sequence encoding a selectable marker conferring drug resistance; and
packaging the targeting vector for delivery to the somatic cell.

8. The method of claim 7, the step of constructing the targeting vector further comprising the steps of:
ligating the delivery vehicle with the modification cassette; and
inserting the polynucleotide sequence encoding an epitope between the first MCS and the selectable marker.

9. The method of claim 8 further comprising the steps of:
preparing first and second homology arms, each of the first and second homology arms comprising a polynucleotide sequence that is homologous to the 5′ and 3′ regions flanking the target gene locus, respectively; and
cloning the first and second homology arms into the first and second MCSs, respectively.

10. The method of claim 9, the step of transfecting a somatic cell with a targeting vector further comprising the steps of:
selecting for a somatic cell which is resistant to a drug;
screening the drug-resistant somatic cell to confirm locus-specific integration of the modification cassette; and
excising the selectable marker conferring drug resistance.

11. The method of claim 10, the targeting vector comprising a recombinant adenoassociated virus (AAV) virion.

12. The method of claim 11, the recombinant AAV virion including first and second inverted terminal repeats (ITRs) linked to the first and second MCSs of the modification cassette, respectively.

13. The method of claim 12, the selectable marker comprising a promoter linked to a polynucleotide encoding resistance to an antibiotic.

14. The method of claim 13, the selectable marker being flanked by lox P sites.

15. The method of claim 14, the epitope comprising three tandem arrayed FLAG epitopes.

16. A method for characterizing an endogenous polypeptide of a somatic cell, the method comprising:
knocking-in an epitope tag-encoding polynucleotide into an endogenous locus of a somatic cell so that an epitope tagged endogenous polypeptide is expressed by the cell; and
immunoprecipitating the tagged polypeptide with an antibody that is specific to the tag; and
characterizing the immunoprecipitated polypeptide.

17. The method of claim 16, the epitope tagged endogenous polynucleotide being knocked-in by homologous recombination mediated knock-in.

18. The method of claim 17, the epitope tag-encoding polynucleotide being knocked-in by transfecting the somatic cell with a targeting vector, the targeting vector including a delivery vehicle linked to a modification cassette, the modification cassette including the epitope tag-encoding polynucleotide.

19. The method of claim 18, the step of transfecting a somatic cell with a targeting vector further comprising the steps of:
constructing the targeting vector to genetically modify an endogenous target gene locus in the somatic cell, the modification cassette further including first and second multiple cloning sites (MCSs) and a polynucleotide sequence encoding a selectable marker conferring drug resistance; and
packaging the targeting vector for delivery to the somatic cell.

20. The method of claim 19, the step of constructing the targeting vector further comprising the steps of:
ligating the delivery vehicle with the modification cassette; and
inserting the polynucleotide sequence encoding an epitope between the first MCS and the selectable marker.

21. The method of claim 20 further comprising the steps of:
preparing first and second homology arms, each of the first and second homology arms comprising a polynucleotide sequence that is homologous to the 5′ and 3′ regions flanking the target gene locus, respectively; and
cloning the first and second homology arms into the first and second MCSs, respectively.

22. A targeting vector for genetically modifying an endogenous target gene locus in a somatic cell, the targeting vector comprising:
a delivery vehicle; and
a modification cassette linked to the delivery vehicle, the modification cassette including a polynucleotide sequences encoding an epitope;
wherein the modification cassette is integrated into the target gene locus by homologous recombination without affecting endogenous transcriptional regulation of the target gene locus.

23. The targeting vector of claim 22, the targeting vector comprising a recombinant AAV virion.

24. The targeting vector of claim 23, the modification cassette further including first and second MCSs and a polynucleotide sequence encoding a selectable marker conferring drug resistance.

25. The targeting vector of claim 23, the recombinant AAV virion including first and second ITRs linked to the first and second MCSs of the modification cassette, respectively.

26. The targeting vector of claim 24, the first and second MCSs having first and second homology arms respectively inserted therein, each of the first and second homology arms comprising a polynucleotide sequence that is homologous to the 5′ and 3′ regions flanking the target gene locus, respectively.

27. The targeting vector of claim 24, the selectable marker comprising a promoter linked to a polynucleotide encoding resistance to an antibiotic.

28. The targeting vector of claim 22, the epitope comprising three tandem arrayed FLAG epitopes.


SEQUENCE LISTING (8 sequences)

SEQ ID NO: 1 - protein, 8 aa, Homo sapiens
DYKDDDDK

SEQ ID NO: 2 - protein, 10 aa, Homo sapiens
EQKLISEEDL

SEQ ID NO: 3 - DNA, 8 bp, Homo sapiens
gggaaagu

SEQ ID NO: 4 - DNA, 8 bp, Homo sapiens
ggagacau

SEQ ID NO: 5 - DNA, 8 bp, Homo sapiens
ggtcccau

SEQ ID NO: 6 - DNA, 8 bp, Homo sapiens
ggcatagu

SEQ ID NO: 7 - DNA, 44 bp, Homo sapiens
tgctgagggaaagtctagaggatcctctagatgtctcctcagca

SEQ ID NO: 8 - DNA, 44 bp, Homo sapiens
cgctgaggtcccatctagaggatcctctagactatgcctcagct